(19) United States
(12) Patent Application Publication
(10) Pub. No.: US 2018/0222836 A1
(43) Pub. Date: Aug. 9, 2018

(54) Process for the Preparation of Pharmaceutical Grade Ferric Citrate

(71) Applicant: Lupin Limited, Mumbai (IN)

(72) Inventors: Anurag Anil Smart, Pune (IN); Yogesh Subhash Aher, Pune (IN); Sunilkumar Vinubhai Gohel, Pune (IN); Rajinder Singh Siyan, Pune (IN); Nandu Baban Bhise, Pune (IN); Girij Pal Singh, Pune (IN)

(21) Appl. No.: 15/749,905

(22) PCT Filed: Aug. 4, 2016
(86) PCT No.: PCT/IB2016/054709

§ 371 Date: Feb. 2, 2018

(30) Foreign Application Priority Data
Aug. 5, 2015 (IN) 2964/MUM/2015

(51) Int. Cl. C07C 51/41; C07C 59/265; A61P 3/12; A61P 7/08; A61P 13/12

(52) U.S. Cl. CPC C07C 51/418; C07C 59/265; C07C 51/42; A61P 7/08; A61P 13/12; A61P 3/12

(57) ABSTRACT

A one-pot process is disclosed for the preparation of pharmaceutical grade ferric citrate that includes preparing a ferric hydroxide slurry followed by treatment with a citrate ion source to yield pharmaceutical grade ferric citrate.

### FIELD OF THE INVENTION

The present invention relates to a one-pot process for the preparation of pharmaceutical grade ferric citrate which is used for the treatment of hyperphosphatemia and metabolic acidosis.

### BACKGROUND

Coordination complexes of ferric citrate and ferric containing compounds are inorganic, iron-based compounds that have the capacity to bind to phosphates and to form non-absorbable complexes with phosphates. Such ferric compounds are useful for the treatment of hyperphosphatemia and metabolic acidosis.

Hyperphosphatemia is associated with severe complications, such as hypocalcemia, decreasing of vitamin-D production, metastatic calcification. Hyperphosphatemia is also contributing to the increased incidence of cardiovascular disease among dialysis-dependent patients and can result in bone pathology.

Studies on the efficacy and tolerability of ferric compounds such as ferric citrate, ferric ammonium citrate and ferric chloride as phosphate binders have been reported in various publications.

Prior studies (U.S. Pat. No. 5,753,706 and J. Am. Soc. Neprol., Vol. 10, Pages 1274-1280, 1999) indicate that ferric compounds are known to bind to dietary phosphate and significantly affect phosphate metabolism.

U.S. Pat. No. 6,903,235 patent discloses a process for the preparation of pharmaceutical grade ferric citrate in solid phase. Further, a method of preparation of pharmaceutical grade ferric citrate by isolating ferric hydroxide is disclosed in U.S. Pat. No. 7,767,851 patent and U.S. Pat. No. 8,093,423 patent for treating disorders related to hyperphosphatemia.

These documents are incorporated herein by reference in entirety for all the purposes.

So there exists a scope to develop an improved, cost effective and plant scalable process for the preparation of pharmaceutical grade ferric citrate. The process needs to consistently produce ferric citrate of the required pharmaceutical grade.

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### OBJECT OF THE INVENTION

Objective of the present invention is to provide a pharmaceutical grade ferric citrate.

A second objective of the present invention is to provide an improved, cost effective and plant scalable process for the preparation of pharmaceutical grade ferric citrate.

A third objective of the present invention is to provide a one-pot process for the preparation of pharmaceutical grade ferric citrate.

Another objective of the present invention is to provide a pharmaceutical grade ferric citrate having BET active surface area in the range of 1-15 m²/gm.

Another objective of the present invention is to provide a pharmaceutical composition comprising a therapeutically effective amount of the pharmaceutical grade ferric citrate and a pharmaceutically acceptable carrier, excipient or diluent.

Another objective of the present invention is to provide a pharmaceutical grade ferric citrate for the treatment and/or prevention of hyperphosphatemia and metabolic acidosis.

### DESCRIPTION OF THE INVENTION

A process for the preparation of pharmaceutical grade ferric citrate of the present invention comprises of combining ferric ion with a base to form of ferric hydroxide slurry which is further treated with citrate ion to yield form the pharmaceutical grade ferric citrate.

According to the present invention, a one-pot process for the preparation of pharmaceutical grade ferric citrate comprising the steps:
(a) combining a ferric ion source, a citrate ion source and a base in water to obtain a mixture;
(b) combining the mixture obtained in step (a) with an organic solvent to yield a precipitate;
(c) isolating the precipitate obtained in step (b); and
(d) drying the precipitate isolated in step (c).

In another aspect of the present invention, a one-pot manufacturing process of pharmaceutical grade ferric citrate comprising the steps:
(a) combining a ferric ion source with a base to form a ferric hydroxide slurry in water;
(b) combining a citrate ion source with the slurry obtained in step (a);
(c) combining with an organic solvent to yield a precipitate;
(d) isolating the precipitate obtained in step (c); and
(e) drying the precipitate isolated in step (d).

According to the present invention, the source of the ferric ion is ferric chloride, ferric nitrate, ferric sulfate or a hydrated form thereof; preferably the source of the ferric ion is ferric chloride hexahydrate and the source of citrate ion is citric acid, its commercially available salts or a hydrated form thereof.

According to the present invention, the base is hydroxides or carbonates of alkali or alkaline earth metals. Alkali carbonates, alkali bicarbonates and alkali metal hydroxides (e.g. of sodium) are preferred. In particular, the base is lithium hydroxide, potassium hydroxide, sodium hydroxide, cesium hydroxide, magnesium hydroxide, sodium bicarbonate, sodium carbonate, lithium carbonate, potassium carbonate, cesium carbonate or magnesium carbonate; in the preferred aspect, the base is sodium hydroxide.

According to the present invention, a molar ratio of the base is used from about 3.0 to 4.0 moles with respect to ferric ion; in the preferred aspect, the base is used from about 3.2 to 3.6 moles with respect to ferric ion.

According to the present invention, the reaction of the ferric ion and the base is performed at about 10 to 50° C., preferably at about 10 to 30° C.

According to the present invention, the ferric hydroxide slurry is combined with water at least three times.

According to the present invention, after combining with water, removing the excess aqueous layer of the ferric hydroxide slurry.

According to the present invention, source of citrate ion and the ferric hydroxide slurry are combined in a molar ratio of citrate ion to ferric ion from about 0.95 to 1.1 moles. Preferably, a molar ratio of citrate ion to ferric ion is used from about 1.0 moles to 1.05 moles.

According to the present invention, the reaction between ferric hydroxide slurry and citrate ion is performed at about 60 to 120° C. temperature, preferably at about 70 to 100° C. temperature.

According to the present invention, the reaction of ferric hydroxide slurry and citrate ion is performed for 1 to 4 hours, preferably for 1 to 2 hours. A clear solution is obtained which is subsequently brought in contact with an organic solvent to precipitate the ferric citrate.

According to the present invention, water miscible organic solvents are used for the precipitation of the ferric citrate. Such water miscible organic solvents are protic solvents, aprotic solvents or a mixture thereof. The preferred protic solvent comprises alcohols such as methanol, ethanol, isopropanol, etc. The preferred aprotic solvent comprises ethers like tetrahydrofuran, 1,4-dioxane, etc.; ketones like acetone, 2-butanone, methyl tertiary butyl ketone, etc.; amides like dimethyl formamide, dimethyl acetamide and other solvents like dimethyl sulfoxide, acetonitrile or a mixture thereof.

According to the present invention, a mixture of acetone and isopropanol is preferred to use for the precipitation of the ferric citrate.

According to the present invention, the obtained precipitate of the ferric citrate is isolated, for example by decantation, filtration, centrifugation, preferably by filtration.

According to the present invention, the precipitate is optionally washed with an organic solvent.

According to the present invention, the product is dried under vacuum at about 20 to 50° C., preferably at about 25 to 35° C. for 5-24 hours, preferably for 5-18 hours, or more preferably for 10-12 hours.

The pharmaceutical grade ferric citrate is optionally purified by using an organic solvent to improve colour and texture.

According to the present invention, the yield of the pharmaceutical grade ferric citrate is in the range of about 0.9 (w/w) to about 1.2 (w/w) with respect to citric acid, preferably about 1.0 (w/w) to about 1.1 (w/w) with respect to citric acid.

According to the present invention, the pharmaceutical grade ferric citrate is brown colored powder.

According to the present invention, the pharmaceutical grade ferric citrate having BET surface area in the range of 1-15 m²/mg. the method of analysis for BET surface are of the ferric citrate is performed as per U.S. Pharmacopeia General Chapter <846>.

According to the present invention, a plant scalable process for the preparation of pharmaceutical grade ferric citrate comprising step of:

(a) combining an appropriate amount of a ferric ion source and a base to form a ferric hydroxide slurry;
(b) combining the ferric hydroxide slurry for at least three times with water;
(c) removing excess water from the ferric hydroxide slurry;
(d) combining citric acid or its hydrated form with ferric hydroxide slurry;
(e) increasing the temperature of the reaction mixture at 80-100° C. and maintaining for 1 to 4 hours to obtain a clear solution;
(f) allowing to cool the solution to 25 to 35° C.;
(g) combining with organic solvent to yield a precipitate;
(h) isolating the precipitate yielded in step (g);
(i) optionally, washing the precipitate with an organic solvent; and
(j) drying the precipitate to maintain the organic volatile impurities as per pharmaceutical product guide line.

The pharmaceutical grade ferric citrate according to the present invention is useful for the treatment and/or prevention of hyperphosphatemia and metabolic acidosis.

This invention further relates to a pharmaceutical composition for treating hyperphosphatemia and metabolic acidosis in the humans, comprising an effective amount of the pharmaceutical grade ferric citrate.

A pharmaceutical composition, for the treatment of hyperphosphatemia and metabolic acidosis, comprising the ferric citrate of the present invention can be administered by orally.

The present invention also provides use of pharmaceutical grade ferric citrate of the present invention in the manufacture of a medicament for treatment of hyperphosphatemia and metabolic acidosis.

A pharmaceutical formulation can be adequately provided in unit dosage form and it can be prepared by any method well-known in the field of pharmaceutical technology. Such method comprises a step of mixing the pharmaceutical grade ferric citrate of the present invention with at least one auxiliary ingredient (e.g., a carrier).

Examples of forms of pharmaceutical formulations (dosage forms) include, but are not limited to, oral formulations, such as tablets, pills, capsules, granules, powders, and suspending agents.

Present invention is further illustrated with the following non-limiting examples.

### EXAMPLE-1

### Preparation of Pharmaceutical Grade Ferric Citrate

To a 100 liter reactor, 3.66 liter water was added and cooled to less than 10° C. and 0.732 kg of NaOH was added to the reaction mass and stirred for 20-30 min to get clear solution. Aqueous solution of ferric chloride (1.542 kg ferric chloride hexahydrate dissolved in 1.82 lit water) was added to the reaction mass at 10-30° C. and stirred for 50-60 min at 25-30° C. (15.5 liter×3) water was added to the reaction mass and stirred for 10-20 min. Stopped the stirring and hold the reaction mass for 25-30 min. The excess aqueous layer was removed. 1.2 kg citric acid was added to the reaction mass. The reaction mass was heated at 80-85° C. and stirred for 1 to 2 hours at same temperature. The reaction mass was cooled to 25-30° C. and filtered. 25.0 lit of acetone and 25.00 lit of isopropanol were added to reaction mass and stirred for 60-90 min. Precipitated solid was filtered, washed with acetone and dried under vacuum at 25-30° C.

Yield: 1.25 kg of the ferric citrate (1.05 w/w)
BET Active Specific Surface Area: 6.51 m²/gm.

### EXAMPLE-2

### Preparation of Pharmaceutical Grade Ferric Citrate

Pharmaceutical grade ferric citrate of example-2 was prepared in the same manner as in example-1 using same batch size.

Yield: 1.3 kg of the ferric citrate (1.08 w/w)
BET Active Specific Surface Area: 6.47 m²/gm.

1. A one-pot process for the preparation of pharmaceutical grade ferric citrate comprising:
(a) combining a ferric ion source, a citrate ion source and a base in water to obtain a mixture;
(b) combining the mixture obtained in step (a) with an organic solvent to yield a precipitate;
(c) isolating the precipitate obtained in step (b); and
(d) drying the precipitate isolated in step (c).

2. The process according to claim-1, wherein the ferric ion source is ferric chloride, ferric nitrate, ferric sulphate or its hydrated form.

3. The process according to claim-2, wherein the ferric ion source is ferric chloride hexahydrate.

4. The process according to claim-1, wherein the base is hydroxides or carbonates of alkali or alkaline earth metals.

5. The process according to claim-4, wherein the base is lithium hydroxide, potassium hydroxide, sodium hydroxide, cesium hydroxide, magnesium hydroxide, sodium bicarbonate, sodium carbonate, lithium carbonate, potassium carbonate, cesium carbonate or magnesium carbonate.

6. The process according to claim-1, wherein the citric ion source is citric acid or its hydrated form.

7. The process according to claim-1, wherein the organic solvent is an alcohol, ether, ketone or a mixture thereof.

8. A pharmaceutical composition comprising the ferric citrate prepared according claim-1 and a suitable carrier.

9. The pharmaceutical composition according to claim-8, wherein said composition is in an orally administrable form.

10. The pharmaceutical composition according to claim-9, wherein the orally administrable form is a tablet, a pill, a capsule, a granule, a powder or a suspension.

11. A method of treating hyperphosphatemia comprising administering ferric citrate prepared according claim-1.

12. A method of treating metabolic acidosis comprising administering ferric citrate prepared according claim-1.

13. A pharmaceutical grade ferric citrate prepared according to claim-1, having BET surface area in the range of 1-15 m²/gm.

14. A pharmaceutical grade ferric citrate, prepared according to claim-1.
